(19) United States
(12) Patent Application Publication
(10) Pub. No.: US 2019/0343451 A1
(43) Pub. Date: Nov. 14, 2019

(54) Bite Force Sensor Assembly and a Method of Diagnosing Bruxism

(71) Applicants: Edwin Bodoni, Centennial, CO (US); Evelyn Bodoni, Centennial, CO (US)

(72) Inventors: Edwin Bodoni, Centennial, CO (US); Evelyn Bodoni, Centennial, CO (US)

(21) Appl. No.: 16/141,570

(22) Filed: Sep. 25, 2018

Related U.S. Application Data
(60) Provisional application No. 62/669,073, filed on May 9, 2018.

(51) Int. Cl. A61B 5/00; A61B 5/22

(52) U.S. Cl. CPC A61B 5/4557; A61B 2090/064; A61B 5/228

(57) ABSTRACT

A relatively low cost sensor assembly that can be used to determine a person's maximum bite force (MBF) is described. The assembly comprises a planar force sensor that is sandwiched between pieces of rigid and compliant plastic that when situated between upper and lower molars and bitten facilitates an electronic signal that can be correlated to a bite force. A user's MBF can utilized to determine to diagnose a person as having bruxism.

### RELATED APPLICATIONS

This application claims priority to and fully incorporates by reference U.S. Provisional Patent Application No. 62/669,073 filed on May 9, 2018 entitled Bite Force Sensor Assembly and Methods of Use to Diagnose Bruxism and having the same inventors as the present application.

### BACKGROUND

Bruxism is a condition characterized by teeth grinding most often experienced while a person sleeps although teeth grinding can also occur while awake. Bruxism can over time comprise the integrity of the affected teeth causing the teeth to lose their protective enamel layer or even crack.

Treating bruxism often comprises fitting a user with a mouth guard that keeps the teeth from the lower teeth separated from the upper teeth. Additionally, the use of mouth guards are known to reduce the frequency and severity of teeth grinding overtime.

Unfortunately, affirmatively diagnosing bruxism after it is suspected can be expensive and time consuming. Traditionally, a sleep study is performed with the person suspected of having bruxism being having plurality of sensors attached to the person's head and body. Typically, such studies are performed at specialized facilities requiring the patient to sleep in an unfamiliar bed while potentially uncomfortable. These studies run the risk of failure if the patient cannot get to sleep for a sufficient period of time. In fact, it is often cheaper and more economical, to treat a patient suspected of having bruxism than definitively diagnose the condition in the first place.

Another means of diagnosing bruxism comprises the use of a disposable bruxism screener, such as the BiteStrip™ device formally manufactured by S.L.P. Ltd. of Germany (up2dent.com). It comprises a small self contained electromyography (EMG) monitor that is affixed to the side of a person's face proximate his/her cheek. As the patient sleeps, the monitor records and analyzes the movement of the patient's jaw muscles while he/she sleeps, determines based thereon whether the patient has bruxism and to what extent, and finally, it provides an outcome indicator on a single digit display. The device has been discontinued by the manufacturer but when available was only good for a single use. While less expensive than a fully instrumented sleep study, the cost of the single use device was not insubstantial.

### BRIEF DESCRIPTION OF THE DRAWINGS

FIG. 1 is a perspective view of an assembled bite force sensor assembly according to one embodiment of the present invention.

FIG. 2 is an exploded perspective view a bite force sensor assembly according to one embodiment of the present invention.

FIG. 3 is a perspective view of the bite force sensor assembly coupled to a controller having a USB connection for interfacing with a personal computer according to one embodiment of the present invention.

FIG. 4 is a perspective view of a fully self contained wireless bite force sensor according to one embodiment of the present invention.

FIG. 5 is a block diagram depicting the operational elements of a bite force assembly according to one embodiment of the present invention.

FIG. 6 is a flow chart detailing the operations involved in diagnosing bruxism according to one embodiment of the present invention.

### DETAILED DESCRIPTION

Embodiments of the present invention comprise a relatively low cost sensor assembly that can be used to determine a person's maximum bite force (MBF) in addition to a method of diagnosing bruxism based on a person's MBF. The assembly makes use of a planar force sensor wherein the sensor's electrical resistance varies depending on the level of force applied to it. The sensor is sandwiched in a laminate comprising a plurality of rigid and compliant plastic pieces, which facilitate the effective transfer of force from a person biting on the assembly when placed between his/her upper and lower molars. The assembly further includes a control circuit (or controller) that measures the resistance and sends a signal to a computer or other device for storage and analysis. Alternatively, the controller may include a microprocessor and/or memory that calculates and stores bite force values based on the measured resistance. The controller typically includes at least one output, which can comprise a wired or wireless interface or in some variations a display.

Embodiments of the sensor assembly can be reusable, partially disposable or intended for use by a single patient. The reusable assemblies can further include disposable thin plastic sheaths that are placed over the portion of the assembly being received in the mouth to isolate and protect the assemblies from direct contact with the patient's saliva and other bodily fluids.

The partially reusable assembly may include a reusable controller but permit the sensor portion to be removed and discarded after each use by a patient. The single patient device can be fully self contained with the intent that after bruxism has been diagnosed it can be disposed of or recycled. Alternatively, the single patient type device can be placed in a patient's file for reuse by the patient in the future to measure a patient's MBF and assess the progress in a treatment regimen.

Embodiments of the present invention also include a methodology of diagnosing bruxism using a bite force sensor, such as embodiments of the sensor assembly described herein. In a study conducted by the inventors, a strong correlation between a person's MBF and bruxism was identified: specifically, MBF values significantly greater than the average MBF for a relevant population subset indicate bruxism.

To diagnose bruxism, a patient bites down on the sensor that is placed between his/her upper and lower molars as hard as possible. The resulting value is recorded. The biting step may be repeated to ensure a maximum value is recorded or to determine an average MBF for the patient. Next, the MBF value for the patient is compared to the average MBF for the relevant population. Based on this comparison a diagnosis is made. If the patient's MBF is a certain amount greater than the relevant average MBF, bruxism is confirmed. The relative MBF may also be used to determine the severity of the patient's bruxism with higher MBF values indicating a more serve affliction. Typically, upon confirmation a treatment plan can be prescribed. At certain times during treatment, additional MBF values for the patient may be obtained to assess the effectiveness of treatment.

### Terminology

The terms and phrases as indicated in quotation marks (“ ”) in this section are intended to have the meaning ascribed to them in this Terminology section applied to them throughout this document, including in the claims, unless clearly indicated otherwise in context. Further, as applicable, the stated definitions are to apply, regardless of the word or phrase's case, to the singular and plural variations of the defined word or phrase.

The term “or” as used in this specification and the appended claims is not meant to be exclusive; rather the term is inclusive, meaning either or both.

References in the specification to “one embodiment”, “an embodiment”, “another embodiment, “a preferred embodiment”, “an alternative embodiment”, “one variation”, “a variation” and similar phrases mean that a particular feature, structure, or characteristic described in connection with the embodiment or variation, is included in at least an embodiment or variation of the invention. The phrase “in one embodiment”, “in one variation” or similar phrases, as used in various places in the specification, are not necessarily meant to refer to the same embodiment or the same variation.

The term “couple” or “coupled” as used in this specification and appended claims refers to an indirect or direct physical connection between the identified elements, components, or objects. Often the manner of the coupling will be related specifically to the manner in which the two coupled elements interact.

The term “directly coupled” or “coupled directly,” as used in this specification and appended claims, refers to a physical connection between identified elements, components, or objects, in which no other element, component, or object resides between those identified as being directly coupled.

The term “approximately,” as used in this specification and appended claims, refers to plus or minus 10% of the value given.

The term “about,” as used in this specification and appended claims, refers to plus or minus 20% of the value given.

The terms “generally” and “substantially,” as used in this specification and appended claims, mean mostly, or for the most part.

Directional and/or relationary terms such as, but not limited to, left, right, nadir, apex, top, bottom, vertical, horizontal, back, front and lateral are relative to each other and are dependent on the specific orientation of a applicable element or article, and are used accordingly to aid in the description of the various embodiments and are not necessarily intended to be construed as limiting.

### An Embodiment of a Bite Force Sensor Assembly

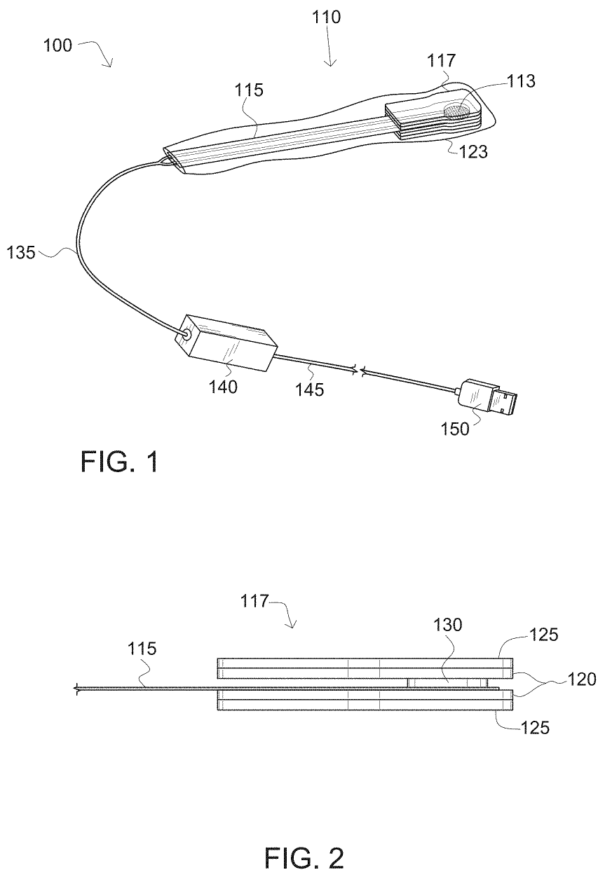

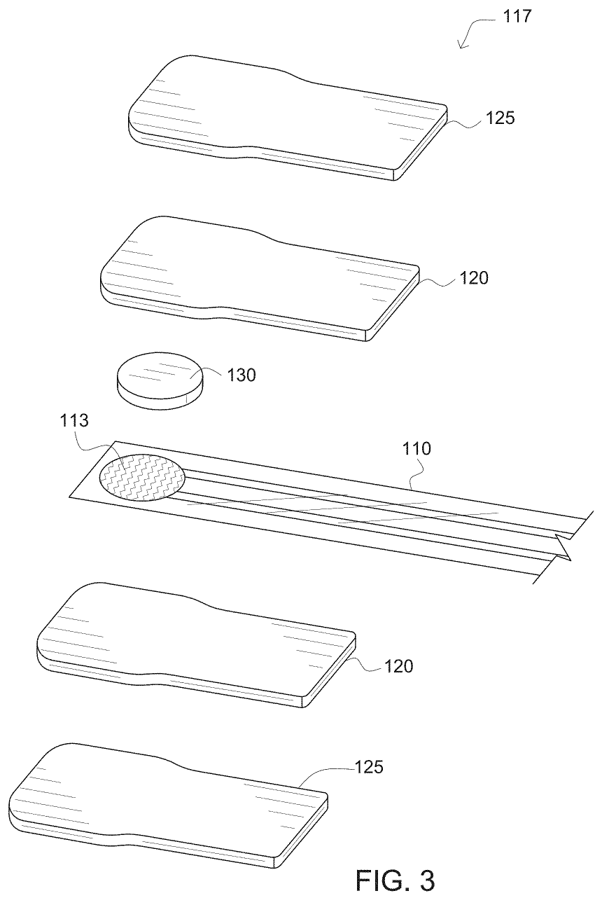

Various views of an embodiment of a bite force sensor assembly are shown in FIGS. 1-3. Generally, the sensor assembly comprises: (1) a flexible force sensor 110 having force sensing portions 113 that are sandwiched in a sensor head subassembly 117 comprising laminate plates 120 & 125 and a force concentrating disk 130; (2) a controller 140 for generating, receiving and transmitting the signal of the force sensing portion; and (3) an output, such as the USB connector 150 shown in FIG. 1.

The force sensor 110 can be any suitable type that is relatively thin and preferably flexible. One sensor type found to be effective is the A201 FlexiForce sensor by Tekscan of South Boston, Mass. The sensor comprises two layers with each layer having a circular sensing portion 113 concentric with and overlaying the other circular sensing portion at the sensor head subassembly 117. When the circular sensing portions are pressed into one another while a current is passed through the sensor, the resistance of the sensor changes relative to the force applied. The measured resistance value can be correlated in to a specific amount of force exerted against the circular sensing portions. As can be appreciated, the sensing portions can be shapes other than circular. The sensor typically includes a long planar lead 115 that is no thicker than the thickness of the stacked circular sensing portions and includes two or more electrically conductive traces. The leads permit the sensor to be coupled with the controller 140.

As can be appreciated different types of force measuring sensors can be utilized other than those relying on a change in resistance to measure force. For instance, a thin flexible capacitive force sensor can be used with appropriate changes to the controller and its programming to accommodate.

The sensor head subassembly 117 as best illustrated in FIGS. 2 & 3 comprises two layer upper and lower laminate sandwiches and at least one force concentrating disk 130. The upper and lower sandwich laminates are essentially identical having a hard inner layer 120 and a soft outer layer 125. These layers are typically adhesively bonded together; however, wherein the layers comprise a similar type of thermoplastic polymeric material, such as EVA or Polyurethane albeit of different hardnesses, the layers can be thermo-bonded together as well. The hard inner layer is configured to help transfer load to the circular sensing portions 113 when the sensor head is bitten by a patient. The soft outer layer is configured to conform to the irregularities of the surface of a patient's upper and lower molars and prevent the biting force from being unduly concentrated on a single part of a molar potentially causing damage to the tooth.

In at least one variation of the sensor head subassembly 117 the hard inner layers comprise 0.040″ thick sheets of rigid Ethylene-vinyl acetate (EVA) plastic preferably having a Shore D hardness of greater than 65. In the same variation, the outer layer also comprises an EVA plastic that is 0.040″ thick but has a Shore A hardness of less than 90.

At least one force concentrating disk 130, typically comprising a rigid piece of plastic, is substantially centered on and placed over at least one side of the circular sensing portions 113 and sandwiched between the upper and lower laminate sandwiches. The force concentrating disk is typically made of a hard material, which can be similar in composition and hardness to the hard inner layers of the laminate sandwiches 120. In other variations, the disk can comprise a different material having a different hardness than the hard inner layers of the laminate sandwiches.

In some variations and as shown in FIG. 2, the disk 130 can be adhesively secured to a respective circular sensing portion 113 on one side and to the respective adjacent upper or lower laminate sandwich on its other side. In the illustrated variation, the opposing circular sensing portion 113 is also adhesively bonded to the other of the upper and lower laminate sandwiches.

In other variations, a circular recess can be provided in an adjacent hard layer 120 to help receive, position and hold the disk 130. In yet other variations, top and bottom disks can be provided instead of one and they both can be received in hard layer recesses and/or adhesively secured to a respective hard layer. In even another variation, a disk like protrusion can be molded on the surface of the hard layer and be fully integral with it.

The diameter of the force concentrating disk 130 is typically no greater and preferably a little smaller in diameter than the diameter of the sensor's circular sensing portions 113 wherein it can be assured that the entirety of the force exerted by a person when biting on the sensor head subassembly 117 is directed through the circular sensing portions. In one variation, the surface area of the force concentrating disk is about 70% that of an adjacent force sensing portions. As can be appreciated, the disk need not be circular as long as the substantial entirety of its surface area that is in contact with the sensor is be contained within the sensor's sensing portions (whether the sensing portions themselves are circular or not).

As shown, the upper and lower laminate sandwiches and the force concentrating disk are held together by way of (i) adhesive bonding of one side of a circular sensing portion 113 to an inside surface of an adjacent hard layer 120, (ii) adhesive bonding of the other side of the sensing portion to the force concentrating disk 130, and (iii) adhesive bonding of the other side of the force concentrating disk to the inside surface of the other hard layer 120. In other variations, any suitable combination of adhesive bonding and mechanical joining can be used to secure the various components together. For instance the upper and lower laminate sandwiches can be mechanically secured together along proximate their respective distal ends at a location wherein they overlie and sandwich the sensor leads 115 but not the circular sensing portions.

With reference again to FIG. 1, the long planar lead 115 of the force sensor 110 terminate at a distal end with the electrical traces of the lead being operatively secured to a cable 135 that extends to a controller 140. The controller measures the magnitude of the resistance and transfers the information in the form of a digital signal to a computing device, such as a personal computer, tablet or smart phone. Depending on the particulars, either the controller or software running on the computing device can calculate the force measurement based on the measured resistance. As shown, the bite force sensor assembly also includes an output cable 145 terminating in a USB connector 150.

In other variations different types of output connectors and cables can be used. Additionally, the output can comprise a wireless transmitter, using a protocol, such as Bluetooth, in place of a wired connection. In yet other variations, the controller can include a WiFi or cellular data transmitter that transmits the acquired data through an internet connection to the computing device or any other suitable device connected to the associated network for processing and data analysis.

As shown in FIG. 1 a disposable sheath 123 can be provided for receipt over the force sensor 110 and the sensor head subassembly 117 to isolate it from direct contact with the oral cavity of a patient, and thereby permit use of the bite force assembly with multiple patients. The sheath typically comprises a sleeve made of polyethylene sheet having a thickness of a few mils or less. The sheath can be removed from the force sensor and disposed of after each use with a new sheath being placed over the force sensor for use with the next patient.

### Another Embodiment of a Bite Force Sensor Assembly

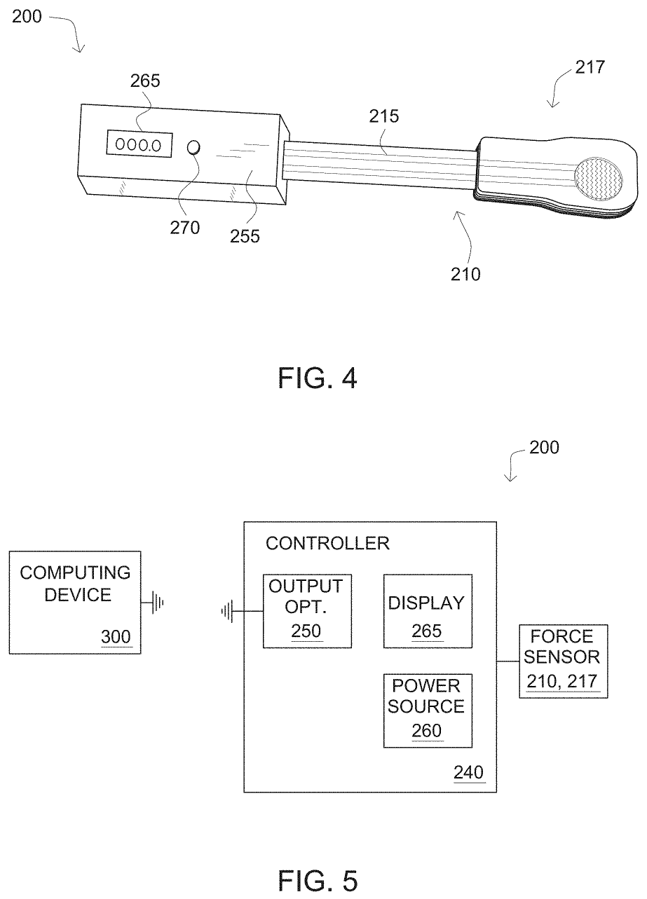

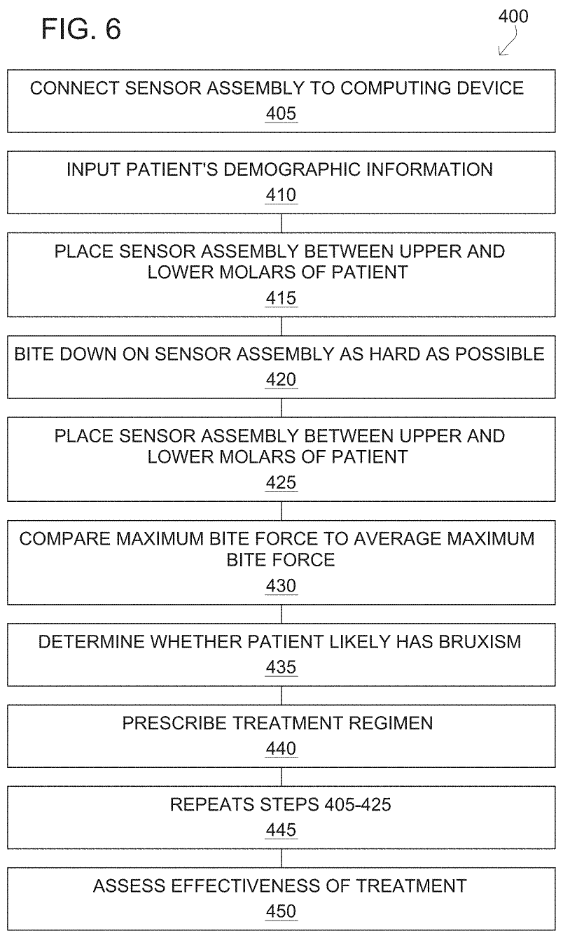

FIG. 4 is a representation of a fully self contained bite force sensor assembly and FIG. 5 is a block diagram showing various necessary and optional operational components of this embodiment. The self-contained bite force sensor assembly 200 includes a force sensor 210 and sensor head subassembly 217 substantially similar to the sensor 110 and sensor head subassembly 117 of the foregoing embodiment of a bite force sensor assembly 100. It further includes a lead 215 similar to the lead 115 of the preceding embodiment albeit shorter. The lead terminates in a body 255 that houses a power source 260, a display 265, an on/off switch 270 and a controller 240. A wireless transmitter 250 can also be provided within the housing to permit the device to transmit the acquired data to a computing device 300. Additionally, although not shown, disposable sheaths may also be provided for use with the assembly.

The controller of the self contained unit will typically include a processor and associated programming to convert the resistance measurement from the sensor to a MBF value which will then be displayed on the display 265 and optionally transmitted wirelessly to a computing device.

### Methods of Diagnosing Bruxism Using a Bite Force Assembly

FIG. 7 is a flow chart illustrating a methodology for diagnosing bruxism using a bite force sensor.

First, as indicated in box 405 and as applicable, the bite force sensor assembly 100, 200 is connected to the associated computing device 300. As necessary, an app or appropriate software program is opened on the computing device. In some variations, connecting the bite force sensor assembly to the computing device can comprise establishing a Bluetooth and/or WiFi connection with the computing device or an associated network. Of note, when using some embodiments, such as the self contained embodiment of FIG. 4, it may not be necessary to connect the sensor assembly to a computing device but rather merely turn it on.

At some point during the process as indicated in box 410, the patient's demographic information is entered into the associated app or program. Demographic information can include, but is not limited to: gender; age, height; weight; and ethnicity. The patient's information is used to more accurately compare his/her MBF data with average MBF values for populations having similar demographic characteristics. The app or software program typically either includes a database of information concerning average MBF values or has access to this information through a internet or other network connection.

After the bite force sensor assembly is connected and activated, the sensor head assembly 117,217 is positioned in the mouth of a patient typically between his/her upper and lower molars as provided in box 415. Next, as indicated in box 420, the patient is directed to bite down on the sensor head subassembly as hard as he/she can. The process of positioning the sensor subassembly and having the patient bite down on it can be repeated several times to ensure the highest possible bite force value can be obtained.

As indicated in box 425, a MBF value is calculated from the resistance value generated as a result of each patient's bite. Depending on the bite sensor assembly, the calculation of the bite force value can be processed by the assembly's controller 140,150 or the resistance value can be transmitted to the computing device wherein the associated app or program calculates the bite force value.

Next as shown in box 430, the patient's MBF value is compared to the average values of people sharing relevant demographic characteristics. This may comprise the app or computer program performing a database look up, or in instances where a standalone device is used, a dentist or other medical professional may look up the average MBF information in printed table or access information available to him/her electronically. A determination is then made by the app or program or the medical professional by comparing the patient's MBF value to an average MBF value whether the patient has or is likely to have bruxism as indicated in boxes 430.

Studies by the inventors have shown a strong correlation between a person's MBF capability and bruxism wherein a MBF significantly greater than the average population of similar demographic characteristics is an accurate means for screening for and even determining whether the patient has bruxism. Specifically, it was determined that men with bruxism had an average MBF value over 50% greater than men without bruxism. For women, those with bruxism had an MBF value over 60% greater than women without bruxism.

The diagnosis of bruxism by a medical professional may take into consideration not only the patient's MBF value but other symptoms reported by the patient or observed in the patient, such as but not limited to, tooth wear from grinding, and jaw pain or soreness.

After a determination has been made that a patient has or is likely to have bruxism, a treatment regimen can be prescribed by the medical professional as indicated in box 440. Treatment may include prescribing a mouth guard that is worn as the patient sleeps.

As shown in block 445, at some point after the treatment has begun, the process of determining the patient's MBF value can be repeated to determine the effectiveness of the treatment. Ideally, if the treatment is effective, the MBF of the patient will be lower than when bruxism was diagnosed. Depending on the results of the follow up determination, the effectiveness of the treatment will be assessed as indicated in box 450, and the prescribed treatment regimen may be continued or modified. Additionally, follow ups MBF values may be obtained on a periodic basis thereafter as deemed prudent or necessary by the medical professional.

### Other Embodiments and Variations

The various embodiments and variations thereof, illustrated in the accompanying Figures and/or described above, are merely exemplary and are not meant to limit the scope of the invention. It is to be appreciated that numerous other variations of the invention have been contemplated, as would be obvious to one of ordinary skill in the art, given the benefit of this disclosure. All variations of the invention that read upon appended claims are intended and contemplated to be within the scope of the invention. For instance, an embodiment of the sensor assembly is contemplated wherein the discreet hard and soft layers are replaced with a single layer having an intermediate hardness, being thicker than the individual hard and soft layers would be, and optionally having a relatively thin soft elastomeric coating on the exterior bite surfaces.

We claim:

1. A bite force sensor assembly comprising:
a thin planar force sensor with (i) a force sensing region having an upper force sensing surface and a lower force sensing surface, and (ii) at least a pair of conductive leads extending outwardly of the force sensing region;
an upper sandwich laminate having an upper outer plate laminated to an upper inner plate wherein the upper inner plate is substantially rigid and the upper outer plate is compliantly resilient;
a lower sandwich laminate having a lower outer plate laminated to an lower inner plate wherein the lower inner plate is substantially rigid and the lower outer plate is compliantly resilient; and
at least one force concentrating portion substantially overlying and in contact with at least one of the upper force sensing surface and the lower force sensing surface;
wherein the force sensor is sandwiched between the upper and lower sandwich laminates.

2. The bite force sensor assembly of 1, wherein the force sensor is flexible.

3. The bite force sensor assembly of 1, wherein the thin force sensor is a resistive sensor with electrical resistance of the force sensor changing depending on a force applied to the force sensing region.

4. The bite force sensor assembly of 1, wherein the upper outer plate and the lower outer plate comprise a polymeric material having a Shore A hardness of less than 90.

5. The bite force sensor assembly of 1, wherein the upper inner plate and the lower inner plate comprise a polymeric material having a Shore D hardness of greater than 65.

6. The bite force sensor assembly of 4, wherein the upper inner plate and the lower inner plate comprise a polymeric material having a Shore D hardness of greater than 65.

7. The bite force sensor assembly of 1, wherein the at least one force concentrating portion comprises a disk, the disk having a Shore D hardness of greater than 65.

8. The bite force sensor assembly of 1, wherein the at least one force concentrating portion is integral with at least one of the upper inner plate and the lower inner plate.

9. The bite force sensor assembly of 1, wherein the at least one force concentrating portion is adhesively bonded to the force sensing region.

10. The bite force sensor assembly of 7, wherein the at least one force concentrating portion is adhesively bonded to at least one of the upper inner plate and the lower inner plate.

11. The bite force sensor assembly of 9, wherein the at least one force concentrating portion is adhesively bonded to at least one of the upper inner plate and the lower inner plate.

12. The bite force sensor assembly of 1, wherein the at least one force concentrating portion overlies and is in contact with at least 70% of an area of the force sensing region.

13. The bite force sensor assembly of 1, wherein the at least one force concentrating portion overlies and is in contact with only the force sensing region of the force sensor.

14. The bite force sensor assembly of 1, in combination with a thin plastic sheath received over the bite force sensor assembly.

15. A bite force sensor assembly comprising:
a flexible thin planar resistive force sensor with (i) a force sensing region having an upper force sensing surface and a lower force sensing surface, and (ii) at least a pair of conductive leads extending outwardly of the force sensing region;
an upper sandwich laminate having an upper outer plate laminated to an upper inner plate wherein the upper inner plate has a Shore D hardness of greater than 65 and a Shore A hardness of less than 90;
a lower sandwich laminate having a lower outer plate laminated to an lower inner plate wherein the lower inner plate has a Shore D hardness of greater than 65 and the lower outer plate a Shore A hardness of less than 90; and
at least one force concentrating disk being in contact with and adhesively bonded (a) to only and at least 70% of the upper force sensing surface of the force sensor and (b) the upper inner plate, the force concentrating disk having a Shore D hardness of greater than 65;
wherein the force sensor is sandwiched between the upper and lower sandwich laminates.

16. The bite force sensor assembly of 1, wherein the upper and lower inner plates and the force concentrating disk comprise a rigid polymeric material and the upper and lower outer plates comprise a compliant and resilient polymeric material.

17. A method of diagnosing and treating bruxism, the method comprising:
placing a bite force sensor assembly between upper and lower molars of a person;
measuring a maximum bite force for the person by having the person bite down on the sensor assembly as hard as he or she can;
comparing the maximum bite force of the person with an average maximum bite force value of a predetermined population having similar demographics to the person; and
based on the comparison wherein the maximum bite force of the person is substantially greater than the average maximum bite force value providing a diagnosis of bruxism.

18. The method of diagnosing and treating bruxism of claim 17 further comprising treating the person for bruxism and thereafter repeating said measuring, and said comparing to determine effectiveness of the treatment.

19. The method of claim 17, further comprising:
inputting demographic information for the person into a computing device having a processor, memory, date storage and at least one input;
operatively coupling the bite force sensor to the computing device through the at least one input; and
the computing device receiving bite force data from the bit force device;
wherein said comparing the maximum bite force of the person is performed on the computing device and includes, (i) using the bite force data to calculate the maximum bite force, (ii) looking up the average maximum bite force value in a database stored in the data storage; and (iii) determining the difference between maximum bite force and the average maximum bite force value.

20. The method of claim 17, wherein said bite force device includes a processor and wherein the processor calculates the maximum bite force value.
